(19) United States
(12) Patent Application Publication
(10) Pub. No.: US 2025/0010100 A1
(43) Pub. Date: Jan. 9, 2025

(54) BRACHYTHERAPY SENSOR CABLE

(71) Applicant: NUCLETRON OPERTIONS B.V., Veenendaal (NL)

(72) Inventors: Cor VAN DE WARDT, Veenendaal (NL); Frits Pieter DE VRIES, Renkum (NL); Johan HENNING, Veenendaal (NL)

(21) Appl. No.: 18/705,893

(22) PCT Filed: Nov. 2, 2022
(86) PCT No.: PCT/NL2022/050617

§ 371 Date: Apr. 29, 2024

(30) Foreign Application Priority Data
Nov. 3, 2021 (NL) 2029601

(51) Int. Cl. A61N 5/10

(52) U.S. Cl. CPC A61N 5/1027; A61N 5/103

(57) ABSTRACT

According to one aspect, a brachytherapy sensor cable is provided which can be moved through a lumen towards an applicator positioned in a human body and wherein a distal part of the sensor cable comprises a sensor; wherein a hollow passage is formed between the proximal part and the distal part to guide signal wiring of the sensor through the hollow conduit of distal part and proximal part towards a connector that is connectable to the afterloader system.

### FIELD OF THE INVENTION

The invention relates to a sensor cable for use with a brachy therapy system and to be used as a check cable in a pre-treatment verification step. The sensor cable performs a check function like obstruction detection, but also can determine channel swap, implant shift, perform reconstruction of implant etc.

### BACKGROUND OF THE INVENTION

In cancer treatment, brachytherapy can be applied interstitially, commonly through afterloading lumens inserted into the target volume. In high dose rate treatment, afterloading catheters are connected to an afterloader, so that a radioactive source can sequentially move to each dwell position inside the target area for a pre-planned dwell time. This process may take approximately 10-20 minutes. Other treatments may involve treatments with applicators that may remain longer in a patient's body, up to several days.

From EP3031494 a verification system is known that can confirm the position of a catheter or lumen inserted within the patient. It includes an elongate control element dimensioned for insertion within the lumen. In a typical brachytherapy treatment method, prior to treatment delivery, one or more lumens, including for example, a brachytherapy applicator, a needle, a tube, or a catheter, are positioned within a target treatment area. The lumen is connected to a source of treatment, and a radiotherapy source is delivered from the treatment source and through the lumen into the treatment area. The lumens are positioned within the patient to deliver the radiotherapy source to suitable, pre-determined treatment locations. The treatment source may be a mechatronic or computerized device (e.g., an afterloader), or the treatment may be delivered manually, and the radiotherapy source may either be a small X-ray generating device, a high dose-rate radioactive source, or a low dose-rate radioactive source for use with longer, shorter, or even permanent dwelling times within the patient.

To increase the effectiveness of brachytherapy, clinicians aim to administer an optimal dosage of radiotherapy source to the target tissue. Following diagnosis, brachytherapy treatment may include multiple stages. Imaging of the patient anatomy and disease anatomy (e.g., tumor location, size, shape, density, orientation) may be analyzed to determine the appropriate regions to administer treatment to. During a treatment preparation and/or planning stage, the desired placement, positioning, and orientation of one or more lumens to deliver the treatment to these target treatment regions may then be determined. Additionally, one or more dwell positions (i.e., locations where the radiotherapy source will remain for a period of time) within each lumen may be mapped in order to achieve a desired dose distribution. During these stages, lumens, which may take the form of an applicator (e.g., having one or more individual lumen channels), needles, tubes, or catheters, may be inserted into a patient, and imaging may be used to confirm the position of the lumens. Next, during a treatment delivery stage, one or more radiotherapy sources may be delivered to the lumens, and the patient may undergo radiation treatment.

Movement or misalignment of one or more lumens may affect the amount of radiation treatment delivered to the target tissue. Misalignment could cause delivery of treatment to the wrong area or delivery of the wrong dosage of treatment to the target area which could arise especially in a treatment procedure that takes longer time, or in which multiple treatments are prescribed over longer time periods. In addition organ swelling may affect the accuracy of lumen positioning. Yet, there is often no convenient way to verify positioning of the lumens after the treatment preparation/planning stage to confirm that the treatment will be delivered as planned.

For example, an applicator may be inserted into a patient for treatment planning, and medical imaging may be used to assess positioning of the lumens. Based on this information, a healthcare provider may determine the location of the dwell positions. Imaging and/or tracking devices and/or processing software may be used to assist with the treatment planning based on the location of the applicator within the body. Once treatment preparation and planning are complete, the patient may be moved into a different room for treatment delivery or otherwise prepared for treatment delivery. The treatment delivery room may include shielding to accommodate use of radioactive materials and may not be compatible with the imaging and/or tracking devices used during treatment planning.

For example, it is important to connect the transfer tube to the correct channel and applicator channel/catheter to assure that the source will be positioned in the intended channel. It would be preferable if channel mapping can be performed automatically.

Also, it would be preferable if a reconstruction of the channel path can be based on imaging performed before the treatment, to define the source path in relation to the target. A treatment plan may be made by automatic reconstruction (contouring) of the catheters/applicator lumens, which is used to calculate dwell positions and times.

Furthermore, it would be preferable if a control system is able to determine if the implant (applicator/catheter) remains at intended positions during the complete treatment, e.g. that may detect shifts of the implanted applicator e.g. due to movement of the patient. These types of treatment may be performed with having multiple treatment channels, wherein at the same time one channel may be checked for starting the radiation treatment, while a next channel may be checked for having a proper position, prior to starting the radiation treatment.

The treatment delivery system (e.g., afterloader) may determine radiotherapy source positioning based on indirect measurements, such as the predetermined dwell positions, saved imaging data, the length of the lumens, the distance that the source has been inserted into the lumens, and the connection of transfer tubes to the lumens. Yet, inaccuracies may occur when relying on secondary measurements. For example, any snaking, bunching, or slack that may be created as a wire with a source or sensor is fed into the lumen may result in inaccurate determinations of how far the source or sensor has been inserted and where in the lumen it is located. Thus, following insertion of the lumen into the body for treatment planning purposes, the lumen may shift within the body, and current systems may not be able to directly determine spatial positioning of the lumen. Consequently, current systems may be unable to directly or accurately verify the ultimate location of the radiotherapy source when delivered to the lumen. Shifting of the lumen after the imaging during treatment planning or preparation may go undetected, resulting in inaccurate radiation treatment for the patient.

It is an object of the present invention to further advance the field of sensor cable devices for use in a brachytherapy afterloader to address these and other challenges.

### SUMMARY OF THE INVENTION

According to one aspect, a brachytherapy sensor cable is provided to be used in a brachy therapy treatment, comprising a hollow conduit in a proximal part of the sensor cable, so that the sensor cable can be moved through a lumen towards an applicator positioned in a human body and wherein a distal part of the sensor cable comprises a sensor; wherein a hollow passage is formed between the proximal part and the distal part to guide signal wiring of the sensor through the hollow conduit of distal part and proximal part. In an embodiment, the signal wiring is guided towards a connector that is connectable to the afterloader system; where it is noted that in using the term ‘afterloading system’ the sensor cable may be used in a verification device separate from an afterloader device, that contains the radio-active source. Such a separate device could perform the actions of verification and planning separately from the afterloader having the radioactive source, which thereby could improve the treatment logistics, since the pretreatment step could be carried out in a separate room with less safety precautions. Such variations are deemed to be encompassed by the scope of the present claims.

It is noted that the term sensor may be used for signal detection, e.g. of an external field, such as an electromagnetic field, or any other field that can be used for the purpose of tracking a treatment path for providing brachytherapy treatment in patient's tissue to be treated. Examples may be measurement of position, radiation, advanced length within the lumen.

The device according to the invention may enable automatic mapping of a channel source path, so that channel swaps can be prevented. Furthermore channel mapping and reconstruction can be provided using the sensor data of the sensor cable and problems due to implant shifts swelling, that could cause a change to the source path can be automatically identified. The sensor is preferably an electromagnetic tracking device, so that no additional ionization radiation is involved. The sensor may additionally be able to detect reference points such as reference marker rings in applicators or markers that identify transfer tube couplings.

### BRIEF DESCRIPTION OF THE DRAWINGS

The invention will be further elucidated in the figures:

FIG. 1 shows a brachytherapy sensor cable is shown according to an embodiment of the invention;

FIG. 2 shows a schematic detail of the distal part, in particular a tip portion without a sensor displayed;

FIG. 3 shows a further schematic detail of the distal part including a sensor;

FIG. 4 provides a detailed view of the connection between the proximal part and the distal part;

FIG. 5 shows an exemplary configuration for a connector;

FIG. 6 shows a schematic overview of a brachytherapy control system;

FIG. 7 depicts a block diagram shown an exemplary workflow interface, according to a further embodiment of the present disclosure;

FIG. 8 depicts a block diagram shown an exemplary workflow interface, according to a further embodiment of the present disclosure; and

FIG. 9 depicts a block diagram shown an exemplary workflow interface, according to a further embodiment of the present disclosure.

### DETAILED DESCRIPTION

Aspects of the invention pertain a brachytherapy sensor cable for connection to a brachytherapy afterloader system to be used in a brachy therapy treatment. The sensor cable comprises a hollow conduit. A proximal part of the sensor cable is connectable to an afterloader system, so that the sensor cable can be moved through a lumen towards an applicator positioned in a human body. A distal part of the sensor cable comprises a sensor; a hollow passage is formed between the proximal part and the distal part to guide signal wiring of the sensor through the hollow conduit of distal part and proximal part towards a connector that is connectable to the afterloader system. In an embodiment the hollow conduit is formed of helical strands.

A proximal part may have a different rigidity to the distal part. At least one of the proximal part and the distal part may comprise a hollow conduit of helical threads. Rigidity is matched to lateral bending stiffness, as flexural rigidity which may be regarded as the is resistance of a conduit against bending deformation.

The helical strands are preferably formed with a stiffness of a first magnitude for the proximal part of the cable; and with a stiffness of a second magnitude, lower than the first magnitude, for the distal part of the hollow conduit. In this way the sensor tip can be moved more flexibly through the brachytherapy channel path which may have a small curvature. This enhances the reliability of the sensor location, since it may be reproduced more easily without shifting of the sensor cable due to snaking. Furthermore, the proximal part may be rendered relatively stiffly, so it can withstand the friction force that will increase towards the proximal position, due to the increased contact of the sensor cable with the conduit wherein it moves.

In order to provide a different flexibility helical strands of proximal part and distal part of the hollow conduit may differ in number. Alternatively or additionally, the number of strands of the distal portion is higher than the number of strands of the proximal part; wherein an increased number of strands may render a more flexible distal portion. In another aspect, the angle or pitch of the helical winding may vary, where a small angle may have an increased rigidity relative to a large angle. The invention is not limited to helical strands but may include strands that are parallel to the cable axis. In a preferred embodiment, the strands are welded together in a metal bushing.

Alternatively or additionally, in order to optimize a desired flexibility characteristic of the hollow sensor cable, the helical strands of proximal part and distal part of the hollow conduit may differ in cross sectional form. For example, a cross sectional form of at least the proximal part may be oblate, to increase the friction force between the strands and thereby increasing the stiffness of at least the lower cable. By suitable combination, the cable can be tuned to have the exact flexibility characteristics of a check cable without a sensor; which check cables are known components that are used to test the clearance of the conduits, before actual administration of the brachy therapy treatment.

A hollow passage may be formed by a weld that connects at least some of the helical strands from the distal part to the proximal part of the cable. In another aspect, the sensor may be a 5-DOF position sensor, i.e. the sensor may have five degrees of freedom.

In yet a further aspect, the sensor is provided in a bushing fixed within to the distal part. In certain embodiments, the sensor comprises a coil that extends in part from the bushing in proximal direction.

A further aspect of the invention pertains to brachytherapy control system, to be coupled to a brachytherapy sensor cable according to any previous claims. The control system may be programmed to process input data from the sensor, to create a three-dimensional channel path profile of a treatment path for providing brachytherapy treatment in patient's tissue to be treated. Furthermore, a treatment and a treatment planning module may be provided for calculating a Biological Effective Dose for brachytherapy treatment, based on the actual registered three-dimensional channel path profile from the sensor registration, of a treatment path for providing brachytherapy treatment in patient's tissue to be treated.

In a further aspect the brachytherapy control system has a treatment planning module provided with:
an image module; arranged to create a three-dimensional channel path profile of a treatment path for providing brachytherapy treatment in patient's tissue to be treated;
a target and organ delineation module; arranged to identify a treatment target and organs at risk in the received image; and
a process module for processing brachy therapy control data in accordance with a treatment plan, and said identified treatment targets and organs at risk.

While in the following description, the invention is embodied with a conduit of helical strands, the invention is not limited to that form but could be carried out with alternative flexible hollow conduit designs, that having a stiffness characteristic that makes them suitable for a brachytherapy sensor cable. For instance the conduit may be formed of braided cable sleeves, or coated flexible conduits, eg helical fibers or strips. Additionally, different material will result in different stiffness characteristic (for example, stainless steel, titanium or nitrol will have varying the stiffness characteristics. Also the number of filars of the hollow helical strand tube will influence stiffness characteristics (higher number of filars gives higher flexibility/lower stiffness).

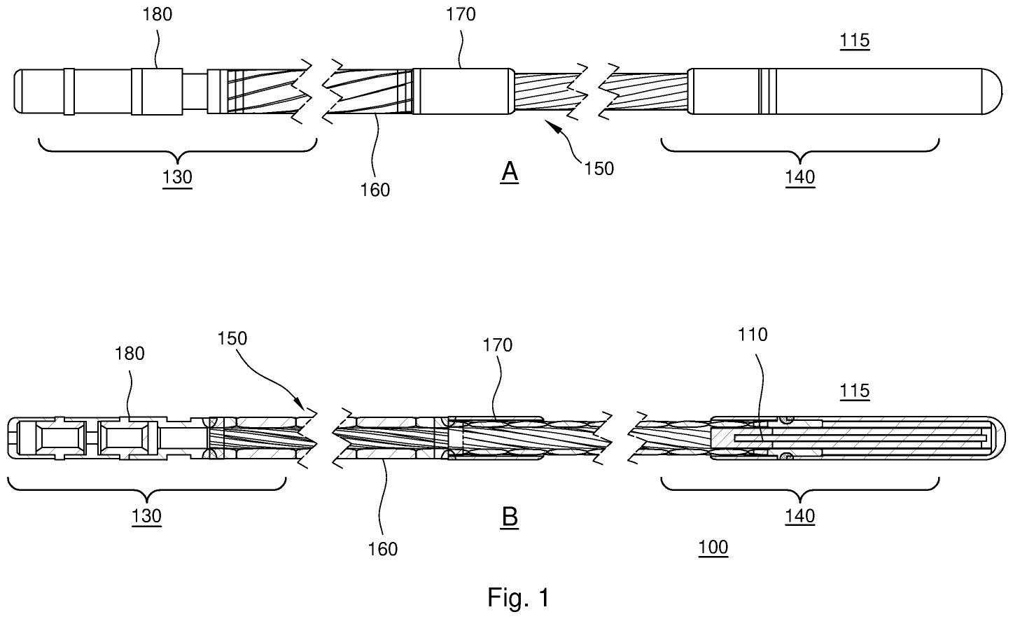

Now turning to FIG. 1 a brachytherapy sensor cable is shown according to an embodiment of the invention. For the sake of simplicity a brachytherapy afterloader system will not be illustrated; but it's connection to the cable will be described in the following. Notably FIG. 1A shows the sensor cable 100 as seen from the outside; FIG. 1B shows interior parts in a cross sectional view. The sensor cable 100 comprises a hollow conduit 150 of helical strands 160, wherein a proximal part 130 of the sensor cable may be connectable to the afterloader system, in the example by a connector 180, to be further described below. The sensor cable 100 can be moved through a lumen towards an applicator positioned in a human body in a way conventional for afterloader systems. The distal part 140 of the sensor cable 100 comprises a sensor 110 in a tip portion 115. A hollow passage 170 is formed between the proximal part and the distal part to guide signal wiring of the sensor through the hollow conduit 150 of distal part and proximal part towards a connector that is connectable to the afterloader system. The passage 170 forms a connection between the proximal part 130 of a first magnitude and the distal part 140 of a second magnitude of the hollow sensor cable 100. While at the proximal part a connector 180 is provided, for the larger part, the proximal part is formed by a hollow helical strand cable, e.g. of about 1 to 4 meters length, e.g. about 2.1 meter and that is guided from the brachytherapy afterloader system through a lumen into a patient's body, as a preceding step prior to the actual radiation step, in order to map the treatment path.

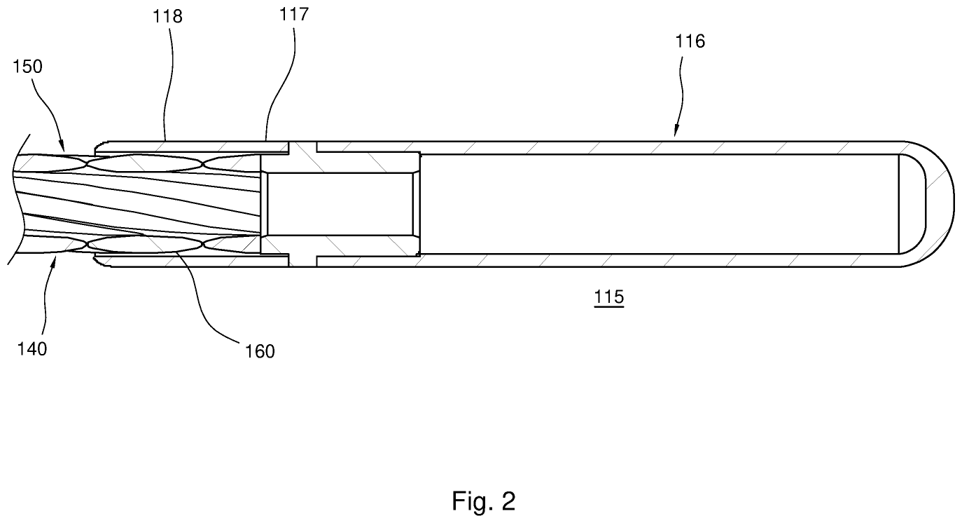

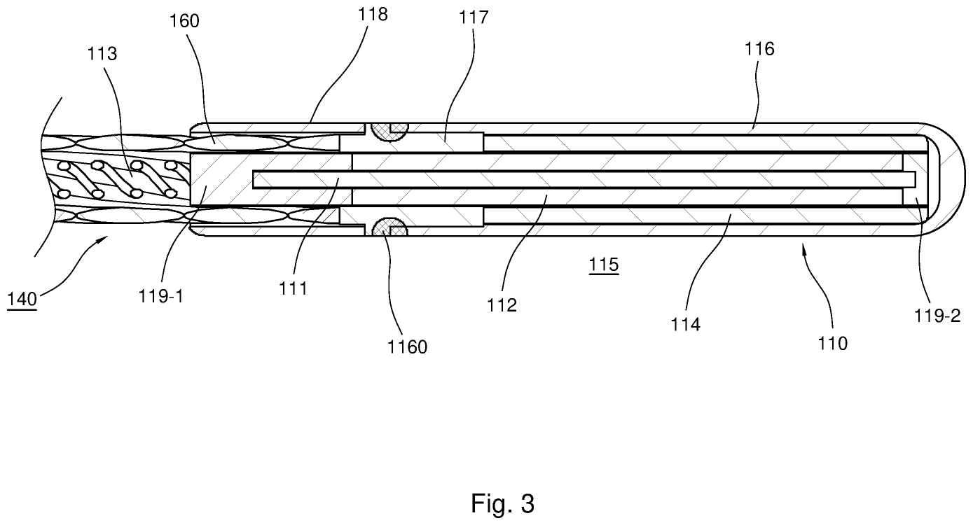

In more detail, FIG. 2 shows a schematic detail of the tip portion 115 of distal part 140. The tip portion 115 comprises a capsule 116 for housing the sensor 110 (see FIG. 3). The capsule 116 has about a maximum diameter corresponding to the diameter of the proximal part 130 of the sensor cable 100, which may be ranging between 0.8 and 1 millimeter, where the distal part 140 has a smaller diameter, e.g. of about 0.7 mm. The capsule 116 is connected to the distal part of hollow helical strand cable 150 via a hollow seat element 117, that forms a passage between an end face of the hollow helical strands 160 of the cable 150, and the capsule 116, that is seated on the seat element 117. Preferably, the hollow seat element 117 is held in a bushing 118, that may be flush with the capsule 116 and that extends from the capsule in proximal direction to provide stiffness to the tip portion 150.

FIG. 3 shows an exemplary embodiment of sensor 110 held in capsule 116. The sensor 110 is a five degree of freedom sensor that may be formed by configuration of a core 111 with windings 112 around it configured to pick up electromagnetic fields. The windings are electrically connected to the connector 180 shown in FIG. 1, by signal wiring, e.g. an electrical lead, such as a twisted copper pair 113 that is guided through hollow cable 140. The sensor 110 may have invariant responses for rotations around the core axis 111, but is able to measure the remaining degrees of freedom adequately in an exterior electromagnetic field. To improve the tracking accuracy the sensor windings 112 are preferably substantially remote from the helical strands 160 and separated by seat 117, which is preferably neutral to the electromagnetic response of the sensor 110. To this end the hollow seat element 117 may be shaped to allow access to both the core 111 and the signal wiring 113 to connect to windings 112. To protect the core from lateral movements of the hollow cable, the sensor 110, in particular the core 111 is axially held by sensor potting pieces 119-1 and 119-2 and may be clamped fixedly in capsule 116 e.g. by a plastic bushing or sensor shaft 114, which may be glued to fit in the space formed by capsule 116. Capsule 116 may be welded by laser welds 1160 to a plug, e.g. seat part 117, so that distal tip portion 115 is substantially rigid without allowing any lateral movement, so that the core 111 is protected from bending or cracking. Beside protecting the sensor 110, the bushing 118 also has the function to protect the laser weld 1160 of the HHS cable 140 to the seat 117.

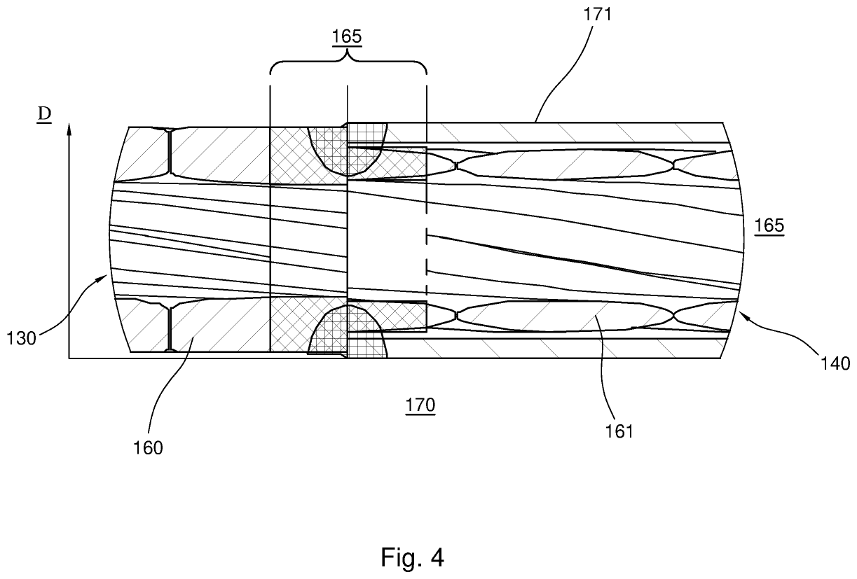

Bushing 118 is a strain relief which prevents fatigue (breaking of laser weld) due to flexing of distal tip when cable is moved through applicator curvatures. Same is applicable for bushing 171 in FIG. 4.

To optimize the robustness and use in the imaging environment, which may be a scanner with a high magnetic field strength e.g. >2 T suitable materials may be stainless steel and polymer, e.g. PEEK, Polyimide to not disturb the sensor signal e.g. by ferromagnetic materials. Because the sensor cable may be held close to a radioactive source, parts of the cable may have to withstand gamma radiation. This is in particular relevant for the sensor consisting of coil, core, potting, shrink tube and twisted pair. Such a radiative transit dose may be received by the cable when it moves beside the radioactive source, but also in stored situation in safe. And for scenario when sensor cable steps through applicator while other lumen is used to irradiate the target.

FIG. 4 provides a detailed view of the passage 170 which forms a connection between the proximal part 130 and the distal part 140 of the hollow sensor cable 100. In welding region 165 helical strands 160 of proximal part 130 are welded together with end faces joining to the helical strands 161 of distal part 140. Proximal strands 160 abut to a bushing 171 that protects distal strands 161 from flexing on the welding region 165. Preferably, bushing 171 is substantially flush with outer diameter D of proximal part 130. Furthermore proximal part 130 is formed with a lateral bending stiffness higher than a lateral bending stiffness of the distal part 140. This provides an advantage for the brachytherapy sensor 110, since the higher stiffness of the larger proximal part limits the ‘snaking effect’. Such a snaking effect occurs when the cable advances in the proximal part of the lumen but without corresponding advance of the cable in the distal part due to bending of the cable. The increased stiffness of proximal part increases a reliability of the advancement measured by the afterloader and the sensor location can be coupled well to the linear advancement of the sensor in the lumen by limiting/minimizing of the snaking effect. To allow for a desired rigidity of the proximal part 130 the helical strands of proximal part and distal part may differ in number with a higher number of strands increasing the flexibility of the distal part 140. Additionally, or alternatively, the helical strands of proximal part 130 and distal part 140 of the hollow conduit 150 may differ in cross sectional form, wherein advantageously a cross sectional form of at least the proximal part is oblate, which increases the stiffness thereof.

This passage 170 is thereby able to have an electrical lead guided through the conduit 165 while having a mechanical characteristic of a conventional brachytherapy wire cable, that advances a radiation source through a lumen, or that advances a ‘dummy’ through the lumen, and that is equally flexible on both proximal and distal parts, thus providing an optimal balance between prevention ‘snaking’, necessary for allowable distance measurement and smooth advancement and flexibility of the tip, also necessary for smooth advancement especially when the lumens for brachytherapy treatment have some bending radius.

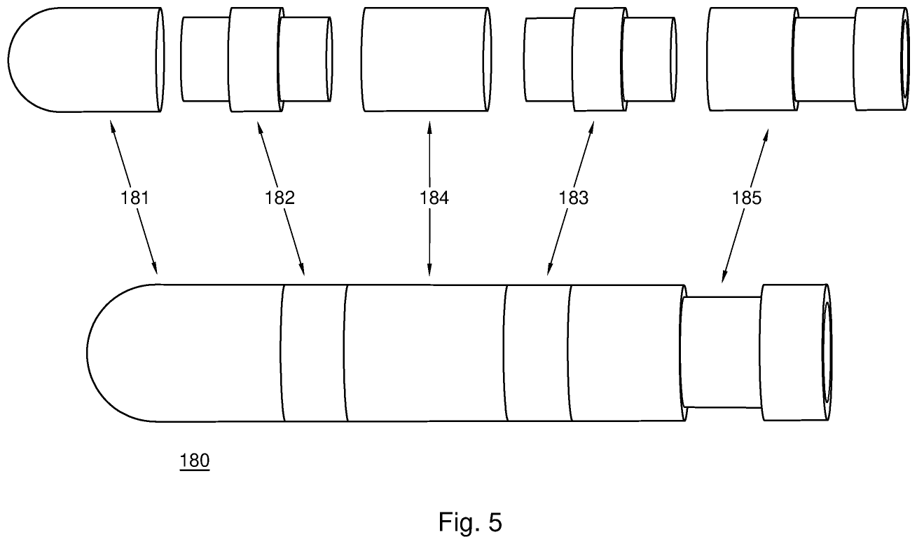

FIG. 5 shows an exemplary configuration for a connector 180, which in this case is a male connector, to be connected to a corresponding female slot provided in the brachytherapy afterloader device, preferably the female slot is provided on a winding coil, so that the sensor cable can be advanced into a lumen for brachytherapy treatment in a conventional way to an appropriate treatment path. In this way, the sensor cable can be electrically connected to a corresponding system, hereafter explained in more detail, and as already described in EP3031494. In the example the connector 180 comprises a conductive connector tip 181, and two conductive annular terminal pieces 184, 185 that are separated from each other by annular insulator parts 182, 183. Terminal pieces 181, 184 are galvanically separated from the proximal conduit 130 by a clamping piece 185 that is fixed, e.g. by laser welding and/or gluing, to proximal conduit 130 (see FIG. 1), and that has a recessed shape to be coupled to a clamping mechanism that fixes the cable into its corresponding female slot. A release mechanism may provide for coupling and releasing the connector, not further illustrated.

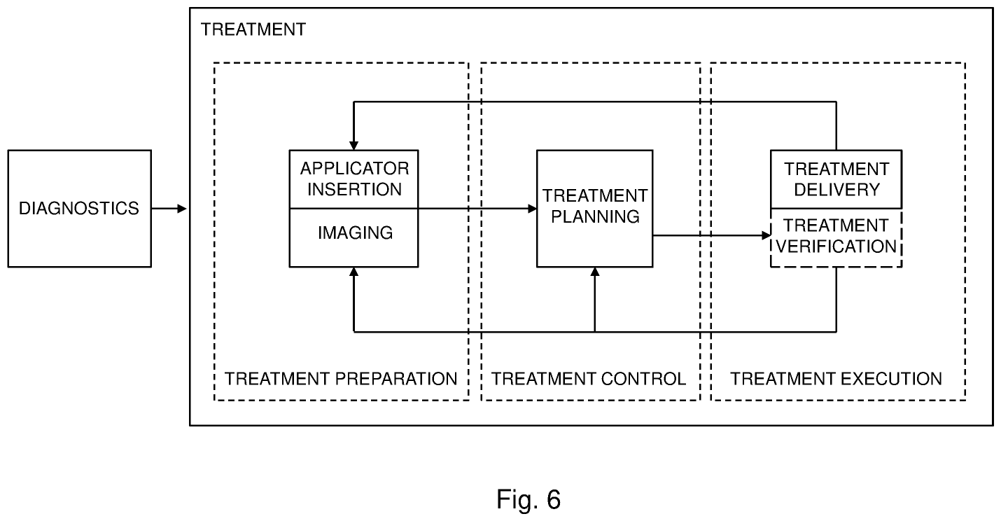

In the remainder, FIGS. 6-9 provide an illustrative system layout of a brachytherapy system, to be used in connection with the sensor cable as presently disclosed in particular, a brachytherapy treatment control system, to be coupled to a brachytherapy sensor cable programmed to process input data from the sensor, to create a three-dimensional channel path profile of a treatment path for providing brachytherapy treatment in patient's tissue to be treated.

As is shown in FIG. 6, the brachytherapy treatment control system may include a treatment and a treatment planning module calculating a Biological Effective Dose for brachytherapy treatment preparation, control, and execution (e.g., one or more of applicator insertion with or without imaging, treatment planning, treatment delivery, with or without treatment verification). During treatment preparation, the conduit may be placed within a patient, and the reference markers associated with the conduit may be detected by a medical imaging system, such as magnetic resonance imaging (MRI), (computed) tomography (CT), (computed) radiography (CR), X-ray, elastography, thermography, photo acoustic imaging, tomography, angiography, optical, near infrared spectroscopy, electromagnetic, nuclear medical, and/or ultrasound imaging. The reference marker detection data may be transmitted to a processor for use with the planning treatment software and/or treatment delivery software. The treatment software analyzes this detection data and determines the two-dimensional or three-dimensional position of the reference marker (and thus conduit) within the patient.

During the treatment control stage, treatment planning may occur, and the precise dose distribution and dwell positioning may be mapped out, based on the positioning of the conduits relative to the patient anatomy. During treatment execution, treatment may be delivered via the conduits, and treatment verification may occur before, during, and/or after treatment delivery. Though the embodiment of FIG. 1 shows a clear, linear division, the steps may be rearranged or repeated as desired; for example, treatment control and/or execution may also include imaging, and treatment control and/or preparation may also include treatment verification. E.g. an image module may be provided; arranged to create a three-dimensional channel path profile of a treatment path for providing brachytherapy treatment in patient's tissue to be treated; a target and organ delineation module may be provided; arranged to identify a treatment target and organs at risk in the received image; and a process module for processing brachy therapy control data in accordance with a treatment plan, and said identified treatment targets and organs at risk. Once in place, the verification element is passed into the one or more conduits, e.g., during the treatment control and/or treatment execution stage. The verification element may be configured to detect the reference markers to determine whether the positioning of the conduit is correct. The path of the verification element through the conduit may mimic the path that the radiotherapy source will take through the conduit, so use of the verification element to determine positioning relative to a reference marker may allow a healthcare provider to determine whether the radiotherapy source will be correctly positioned. Exemplary embodiments of this system are described in further detail below.

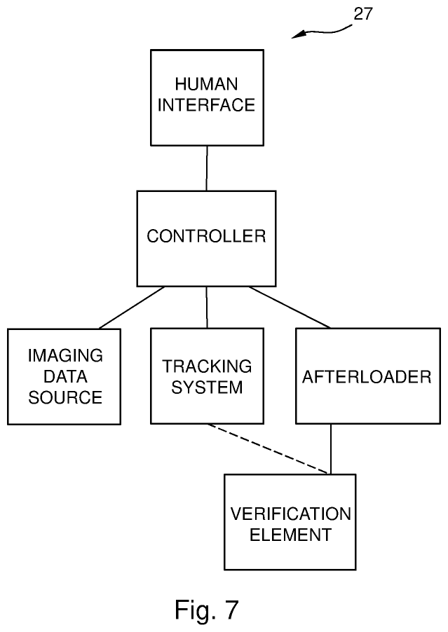

FIGS. 7, 8, and 9 provide an overview of exemplary brachytherapy systems that use a verification element to detect the location of conduits that will be used to deliver treatment in order to promote more accurate dose delivery. In the embodiment of FIG. 7, the verification element communicates with an afterloader regarding the detected position of one or more reference markers, which may be used to determine the location of one or more conduits. A tracking system may also communicate with the verification element in order to determine the location of the reference marker(s). Based on this information, system 27 may determine whether the conduits are located in the intended location, or, if there is a deviation from the intended location, whether the actual location is within an acceptable threshold of deviation. To help in this determination, the controller may communicate with an imaging data source to compare the detected location of the conduits with an image of the conduits within the body or to compare the detected location of the conduits with an image of the surrounding body structures. The image may have been taken at an earlier step, e.g., during treatment preparation or planning, or may be taken during the verification procedure. A controller may act as the interface between the various components of system 27, controlling communications between the components and/or controlling the actions of one component based on signals received from that component, another component, or based on data received from multiple components.

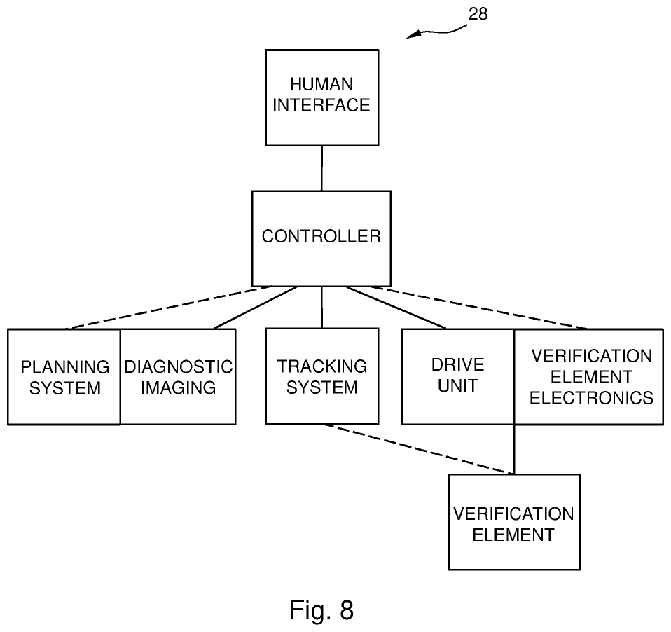

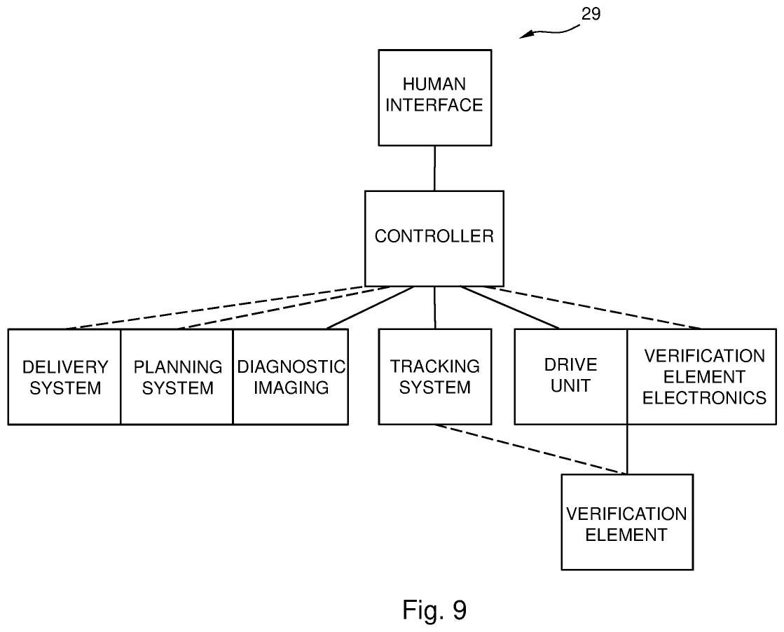

As is shown in FIG. 8, a system 28 may include a drive unit and verification element electronics for controlling movement of the verification element relative to the conduits and/or reference markers. These components may be independent from an afterloader device, or may be included in an afterloader device, and may work with a tracking system to determine the location of the verification element relative to the reference marker(s). All three components may be in communication with the verification element, and the controller may adjust the drive unit or the electronics based on information gleaned from the verification element, the tracking system, the drive unit, or the verification element electronics. Based on this information, system 28 may determine whether the conduits are located in the intended location, or, if there is a deviation from the intended location, whether the actual location is within an acceptable threshold of deviation. To help in this determination, the controller may communicate with an imaging data source, as describe in system 27 of FIG. 7. Further, the controller may communicate with a planning system to further determine whether the intended dose distribution will be achieved within an acceptable threshold based on the actual location of the reference markers and conduits that is detected by the verification element. Additionally, as is shown in system 29 of FIG. 9, the controller may also communicate with a delivery system to adjust the actual delivery of treatment, if desired, based on the actual location information detected.

It is thus believed that the operation and construction of the present invention will be apparent from the foregoing description and drawings appended thereto. For the purpose of clarity and a concise description, features are described herein as part of the same or separate embodiments, however, it will be appreciated that the scope of the invention may include embodiments having combinations of all or some of the features described.

The sensor cable may applied wherever HDR-BT or PDR-BT is considered a beneficial procedure to apply ionising radiation in the process of cancer treatment. Among others: Prostate Cancer, Glioblastoma Multiforme cancer treatment, Gynaecological cancer treatment, Rectal cancer treatment, Breast cancer treatment, Head & Neck cancer treatment, etc.

It will be clear to the skilled person that the invention is not limited to any embodiment herein described and that modifications are possible which may be considered within the scope of the appended claims. Also kinematic inversions are considered inherently disclosed and can be within the scope of the invention. In the claims, any reference signs shall not be construed as limiting the claim. The terms ‘comprising’ and ‘including’ when used in this description or the appended claims should not be construed in an exclusive or exhaustive sense but rather in an inclusive sense. Thus expression as ‘including’ or ‘comprising’ as used herein does not exclude the presence of other elements, additional structure or additional acts or steps in addition to those listed. Furthermore, the words ‘a’ and ‘an’ shall not be construed as limited to ‘only one’, but instead are used to mean ‘at least one’, and do not exclude a plurality. Features that are not specifically or explicitly described or claimed may additionally be included in the structure of the invention without departing from its scope. Expressions such as: “means for . . . ” should be read as: “component configured for . . . ” or “member constructed to . . . ” and should be construed to include equivalents for the structures disclosed. The use of expressions like: “critical”, “preferred”, “especially preferred” etc. is not intended to limit the invention. To the extent that structure, material, or acts are considered to be essential they are inexpressively indicated as such. Additions, deletions, and modifications within the purview of the skilled person may generally be made without departing from the scope of the invention, as determined by the claims.

1. A brachytherapy sensor cable wherein the sensor cable comprises a hollow conduit in a proximal part of the sensor cable so that the sensor cable can be moved through a lumen towards an applicator positioned in a human body and wherein a distal part of the sensor cable comprises a sensor; wherein a hollow passage is formed between the proximal part and the distal part to guide signal wiring of the sensor through the hollow conduit of distal part and proximal part.

2. A brachytheraby sensor cable according to any preceding claim, wherein the proximal part has a lateral stiffness higher than the lateral stiffness of the distal part.

3. A brachytherapy sensor cable according to claim 2, wherein the proximal part comprises a conduit of helical strands.

4. A brachytherapy sensor cable according to claim 2 or 3, wherein the distal end comprises a conduit of helical strands.

5. A brachytherapy sensor cable according to claim 4 when appended to claim 3 wherein the number of strands in the distal end is greater than the number of strands of the proximal part.

6. A brachytherapy sensor cable according to claim 5, or claim 4 when appended to claim 3 wherein the strands in the distal end is of a different material to the strands of the proximal part

7. A brachytherapy sensor cable according to any one of claims 4 to 6, wherein said distal strands are enclosed by a bushing that is welded to the proximal part.

8. A brachytherapy sensor cable according to any preceding claim 2-7, wherein, the helical strands of proximal part and distal part of the hollow conduit differ in cross sectional form.

9. A brachytherapy sensor cable according to any preceding claim, wherein a cross sectional form of at least the proximal part is oblate.

10. A brachytherapy sensor cable according to any previous claim wherein the hollow passage is formed by a weld that connects at least some of the helical strands from the distal part to the proximal part of the cable.

11. A brachytherapy sensor cable according to any previous claim, wherein the sensor is a 5-DOF sensor.

12. A brachytherapy sensor cable according to any previous claim, wherein the sensor is provided in a capsule fixed to the distal part.

13. A brachytherapy sensor cable according to claim 11, wherein the capsule is of stainless steel, and wherein the sensor is provided in a polymer shaft embedded in the capsule.

14. A brachytherapy sensor cable according to any previous claim, wherein a proximal part of the sensor cable is connectable to an afterloader system to be used in a brachy therapy treatment.

15. A brachytherapy control system, to be coupled to a brachytherapy sensor cable according to any previous claims, said control system programmed to process input data from the sensor, to create a three-dimensional channel path profile of a treatment path for providing brachytherapy treatment in patient's tissue to be treated.

16. The brachytherapy control system according to claim 15, further comprising a treatment and a treatment planning module calculating a Biological Effective Dose for brachytherapy treatment.

17. The brachytherapy control system according to claim 16, wherein the treatment planning module is provided with:
an image module; arranged to create a three-dimensional channel path profile of a treatment path for providing brachytherapy treatment in patient's tissue to be treated;
a target and organ delineation module; arranged to identify a treatment target and organs at risk in the received image;
a process module for processing brachy therapy control data in accordance with a treatment plan, and said identified treatment targets and organs at risk.
